(19) United States
(12) Patent Application Publication
(10) Pub. No.: US 2024/0164665 A1
(43) Pub. Date: May 23, 2024

(54) SYSTEM AND METHOD FOR ASSESSMENT OF STROKE PATIENTS AND PERSONALIZED REHABILITATION

(71) Applicant: University of Pittsburgh - Of the Commonwealth System of Higher Education, Pittsburgh, PA (US)

(72) Inventors: Amit Sethi, Pittsburgh, PA (US); Marcus C. Allen, Robbinsville, NJ (US); William W. Clark, Wexford, PA (US)

(73) Assignee: University of Pittsburgh - Of the Commonwealth System of Higher Education, Pittsburgh, PA (US)

(21) Appl. No.: 18/548,875

(22) PCT Filed: Mar. 2, 2022
(86) PCT No.: PCT/US2022/018466

§ 371 Date: Sep. 1, 2023

Related U.S. Application Data
(60) Provisional application No. 63/155,482, filed on Mar. 2, 2021.

(51) Int. Cl. A61B 5/11; A61B 5/00; G16H 10/60; G16H 50/20

(52) U.S. Cl. CPC A61B 5/1124; A61B 5/1127; A61B 5/7405; A61B 5/742; G16H 10/60; G16H 50/20; A61B 2562/0219; A61B 2562/16

(57) ABSTRACT

A system for classifying the severity of a user's impairment can include a target apparatus, a plurality of inertial measurement units, and a processor. The target apparatus can include a base, a target structure coupled to the base, and a plurality of targets coupled to the target structure. The processor can be configured to classify the severity of the user's impairment based on the data collected from the inertial measurement units. A method can include classifying a severity of a person's impairment based on one or more motion features associated with the person's performance of one or more tasks at a target apparatus. A target apparatus used in assessing physical impairment of a user can include a track system, a target structure coupled to the track system, and a plurality of targets coupled to a central portion and arms of the target structure.

### CROSS REFERENCE TO RELATED APPLICATION

This application claims priority to U.S. Provisional Patent Application No. 63/155,482, filed Mar. 2, 2021, which is incorporated herein by reference in its entirety.

### FIELD

The present disclosure relates to systems and methods for stroke diagnostics and task-oriented rehabilitation.

### BACKGROUND

Each year, an estimated 795,000 individuals within the United States suffer from a stroke. Of these individuals, eighty-five percent of them suffer some form of impairment, often having difficulty moving an arm, hand, or other extremity. These adverse effects can have a staggering impact on the quality of one's life. For instance, the inability to move one's hand or arm freely can make performing basic daily tasks, such as eating, dressing, and driving, incredibly difficult. For this reason, stroke is considered one of the leading causes of permanent disability in the United States. Treatment for these impairments involve physical rehabilitation, and evidence suggests that gains in upper extremity function improve with increasing rehabilitative intervention. However, intensive rehabilitative intervention requires extensive outpatient therapy, making at home, self-therapy an ineffective alternative for many. For instance, an estimated sixty-five percent of individuals are unable or unwilling to perform and adhere to a prescribed rehabilitation training outside in-person therapy. To further complicate matters, individuals often avoid seeking treatment altogether due to the high costs involved in seeking treatment or the inability to find a clinic that provides the necessary outpatient services. Thus, an improvement in the field is needed to promote and increase the quality of and accessibility to the rehabilitative intervention stroke patients need to improve mobility.

### SUMMARY

According to an aspect of the present disclosure, a representative embodiment of a system for classifying the severity of a user's impairment can include a target apparatus, a plurality of inertial measurement units, and a processor including computer-readable instructions. The target apparatus can include a base, a target structure coupled to the base, and a plurality of targets coupled to the target structure. Each of the targets are associated with one or more user tasks. The inertial measurement units can be configured to collect data associated with a user's movement as the user performs one or more of the user tasks. By executing the instructions, the processor can be configured to classify the severity of the user's impairment based on the data collected from the inertial measurement units.

In some embodiments, by executing the instructions, the processor can be configured to assign a difficulty ranking to one or more user tasks performed by the user based on the data collected from the inertial measurement units. In further embodiments, by executing the instructions, the processor can be configured to recommend one or more user tasks based on the difficulty rankings assigned.

In some embodiments, by executing the instructions, the processor can be configured to transmit the classification of the user's impairment to a remote device, the remote device being configured to modify the classification based on an input from an operator of the remote device to form a modified classification and communicate the modified classification to the processor. In other embodiments, by executing the instructions, the processor can be configured to transmit the recommended tasks to a remote device, the remote device being configured to modify one or more of the recommend tasks based on an input from an operator of the remote device to form one or more modified recommended tasks and communicate the modified recommended tasks to the processor.

In some embodiments, wherein by executing the instructions the processor can be configured to classify the severity of the user's impairment for each task the person performs at the target apparatus. In such embodiments, a final classification of the severity is an average and/or weighted average of the classifications for the tasks.

In some embodiments, the targets can be arranged in a radial configuration. In other embodiments, one or more of the targets can include an accelerometer, a gyroscope, a magnetometer, or a combination thereof. In still further embodiments, one or more targets can include a sensor configured to detect whether the user task associated with the target has been performed.

In some embodiments, the base can be slidably adjustable relative to the user such that the base is configured to move toward and away from the user. In some embodiments, the target structure can comprise one or more optical devices, audio devices, or a combination thereof to direct the user to perform the user task. In additional embodiments, the system can further include one or more optical tracking systems to collect data associated with user's movement. In still further embodiments, by executing the instructions, the processor can be configured to classify the severity of the user's impairment based on the data collected from the inertial measurement units and the optical tracking systems.

In additional embodiments, by executing the instructions the processor can be configured to recommend one or more user tasks based on the classification of severity.

In some embodiments, one or more of the targets can comprise a platform having one or more sensors configured to detect whether an object positioned on the platform has been moved.

In another representative embodiment, a method can include classifying a severity of a person's impairment based on one or more motion features associated with the person's performance of one or more tasks at a target apparatus, wherein the motion features are determined from data collected from one or more inertial measurement units.

In some embodiments, the severity of the person's impairment can be classified as one of a healthy population, a mildly impaired population, a moderately impaired population, or a severely impaired population. In further embodiments, classifying the severity of the person's impairment can include classifying the severity of the person's impairment for two or more tasks the person performs at the target apparatus, and wherein a final classification of the severity is an average and/or weighted average of the classifications for the two or more tasks.

In some embodiments, the method can include assigning a difficulty ranking to one or more tasks performed by the person at the target apparatus based on the motion features. In some embodiments, assigning a difficulty ranking to a task can comprise determining a deviation between the motion features of the person and one or more motion features of a healthy population for the respective task. In still further embodiments, the method can include recommending one or more tasks to the person based on the difficulty ranking.

In some embodiments, the target apparatus can include a target structure and a plurality of targets coupled to the target structure, each target being associated with one or more of the tasks.

In additional embodiments, the method can use one or more machine-learning methods selected from a perceptron, Bayesian, logistic regression, K-nearest neighbor, neural network, deep learning, and/or a support vector machine algorithm. In still further embodiments, the method can use a support vector machine learning algorithm.

In another representative embodiment, a target apparatus used in assessing physical impairment of a user can include a track system, a target structure coupled to the track system and comprising a central portion and a plurality of outwardly extending arms circumferentially spaced along a circumference of the central portion, and a plurality of targets coupled to the central portion and arms of the target structure, each target being associated with a physical task and configured to couple and decouple to the target structure such that each target can be positioned at various lengths relative to the central portion. The track system can be configured to slidably adjust such that the target structure can be adjusted toward and away from a user.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 is a flowchart illustrating a process for classifying the severity of a user's impairment and recommending tasks for rehabilitation.

FIG. 1B is a schematic diagram depicting a system for classifying the severity of an individual's impairment.

FIG. 2A is a front view of a target apparatus used in task-based testing.

FIG. 2B is a perspective view of the target apparatus of FIG. 2A.

FIG. 2C is a side view of the target apparatus of FIGS. 2A-2B.

FIGS. 3A-3B show inertial measurement units (IMUs) secured to the upper extremities and torso of a user.

FIG. 4 is a flowchart illustrating a method for training a machine-learning algorithm for classifying the severity of a user's impairment.

FIG. 5 is a flowchart illustrating a method for classifying the severity of a user's impairment.

FIG. 6 is a flowchart illustrating a method for difficulty ranking and recommending tasks for rehabilitation.

FIG. 7 is a schematic diagram of an example computing system in which described embodiments can be implemented.

FIG. 8 is a schematic diagram of an example cloud computing environment that can be used in conjunction with the technologies described herein.

### DETAILED DESCRIPTION

### General Considerations

For purposes of this description, certain aspects, advantages, and novel features of the embodiments of the inventive technology are described herein. The disclosed methods, apparatus, and systems should not be construed as being limiting in any way. Instead, the present disclosure includes lower all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

Although the operations of some of the disclosed embodiments are described in a particular, sequential order for convenient presentation, this manner of description encompasses rearrangement, unless particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not excluded the presence of intermediate elements between the coupled or associated items absent specific contrary language.

As used in this application, the term “and/or” used between the last two of a list of elements any one or more of the listed elements. For example, the phrase “A, B, and/or C” means “A,” “B,” “C,” “A and B,” “A and C,” “B and C,” or “A, B, and C.”

In some examples, values, procedures, or apparatus are referred to as “best,” “optimal,” “easiest,” or the like. Such descriptions are intended to indicate that a selection among many used functional alternatives can be made, and such selections need not be better, easier, smaller, or otherwise preferable to other selections.

For the sake of presentation, the description sometimes uses terms like “provide” or “use” to describe the disclosed methods, including computer operations in a computing system. These terms are high-level abstractions of the actual operations that are performed. The actual operations that correspond to these terms may vary depending on the particular implementation and are readily discernible by one of ordinary skill in the art.

Any of the computer-based methods can be implemented as computer-executable instructions or a computer program product stored on one or more computer-readable storage media and executed on a computing device (e.g., any available computing device, including smart phones or other mobile devices that including computing hardware). Computer-readable storage media can include any tangible media that can be accessed within a computing system (e.g., one or more optical media discus such as DVD or CD, volatile memory components (such as DRAM or SRAM), or nonvolatile memory components (such as flash memory solid state drives, or magnetic media such as hard drives)).

Any of the computer-executable instructions for implementing the disclosed techniques as well as any data created and used during implementation of the disclosed embodiments can be stored on one or more computer-readable storage media. The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or other such network) using one or more network computers.

The disclosed technology is not limited to any specific computer language or program. For instance, the disclosed technology can be implemented by software written in C++, Java, Perl, JavaScript, Python, R, Julia, LISP, assembly language, or any other suitable programming language. Likewise, the disclosed technology is not limited to any particular computer or type of hardware.

Furthermore, any of the software-based embodiments (comprising, for example, computer executable instructions for causing a computer to perform any of the disclosed methods) can be uploaded, downloaded, or remotely accessed through a suitable communication means. Such suitable means include, for example, the Internet, the World Wide Web, an intranet, software applications, cable (including fiber optic cable), magnetic communications, electromagnetic communications (including RF, microwave, and infrared communications), electronic communications, or other such communication means.

Introduction to the Disclosed Technology Individuals who experience a stroke suffer from one or more of three motor deficiencies. These motor deficiencies include weakness, dyscoordination (e.g., the inability to effectively synchronize muscle efforts in each limb to complete a task), and hypertonia or reflexia (e.g., tightness of muscle tone and overresponsive reflexes). The current standard to evaluate these deficiencies, such as dyscoordination and weakness, and to assess stroke severity is the Fugl-Meyer Upper Extremity (FMUE) scoring system. A patient's stroke severity using the FMUE system, for example, can be calculated by measuring the patient's range of motion via a goniometer, grip strength by how they grasp and tug on an object, and their speed of motion by measuring the time it takes for the index finger of their impacted armed to touch their knee and then back to their nose.

Although the FMUE scoring system is the standard in measuring motor impairment, it fails to observe dynamic compensatory motor strategies in between the start and end positions of a patient's motion while performing FMUE tasks. For example, the above-mentioned motor deficiencies can cause spatiotemporal irregularities in limb movement during daily living activities, which are difficult to observe qualitatively and can cause further injuries if not corrected.

Additional challenges to stroke recovery and rehabilitation can include limited one-on-one interaction with a clinician, the limitations clinicians face in diagnosing the severity of an individual's impairment, and the difficulty many individuals have in properly following a rehabilitation plan on their own and outside of a clinical setting. For example, clinicians are generally limited in both the time they have with a patient and the movements they can measure in a patient. Clinicians, for instance, are often only able to measure the range of motion of a single joint at a time with a goniometer. This limitation in measurement hinders a clinician's ability to measure dynamic movement while the patient performs functional tasks and can present challenges in establishing optimal rehabilitation goals for patients. Moreover, once diagnosed, patients suffering from some physical and/or neurological impairment are often prescribed a series of exercises to complete on their own and at home, to help in their recovery. However, outside of the clinic and without guidance of a clinician, patients tend to perform exercises incorrectly and become unmotivated, ultimately to the detriment of their own recovery. Incorrectly performing exercises, for example, can reinforce negative compensatory movements and lead to further complications, including further physical impairment and an increase in pain. Significant setbacks in recovery can occur under these circumstances as the quantity and quality of these exercises have been directly linked to the rate and level of recovery.

Other barriers to recovery also include the prohibitive costs of outpatient therapy and the limited number of outpatient clinics that cater to the rehabilitation needs of stroke patients, especially in suburban and rural areas. These barriers for instance, often cause those in need of therapy to end their treatment early in recovery, or avoid seeking treatment at all, making it increasingly likely that these individuals experience further physical impairment and pain, without regaining lost mobility.

Accordingly, innovations that can classify the severity of a patient's impairment initially and/or throughout the process of recovery and rehabilitation locally or remotely can offset the need for clinical interaction and make resources more accessible, such as for at home use. Such innovations can also measure dynamic motion of patients, to classify the severity and communicate both the measurements and severity to a medical professional, increasing the overall quality and efficiency of treatment provided. These innovations can also provide valuable instruction and support to individuals at home through recommending rehabilitation tasks remotely. System and method innovations can in this way, be employed to remotely assess and provide personalized rehabilitation, and a higher-quality therapist guided treatment outside a clinic and in the safety and convenience of the patient's home.

Described herein are systems, methods, and apparatuses for classifying the severity of a user's impairment (also referred to as “impairment severity”) and providing a hierarchy of tasks to the user based on a deviation between their movement and movement of a healthy population. The severity classification is based on a variety of motion features associated with the user's movement and measured as the user moves about a three-dimensional space provided by a target apparatus. The target apparatus can be adjusted relative to the user and comprises a plurality of individual targets that may be adjusted as well. Each of the targets correspond to a given task of a task-based test. The task-based test for instance, can include a series of physical tasks assigned to the user such that the user moves about the space of the target apparatus while performing each of the assigned tasks. As the user moves within the space of the target apparatus to perform each task, one or more measurement devices secured to the user's body collects data for one or more motion features associated with the user's body movement to be calculated and/or extracted. The one or more motion features are then used to classify or predict the severity of the user's impairment via one or more machine-learning tools. Such motion features of one or more classes or populations may also be used to train or test the machine-learning tools. The motion features of the user and/or classes can also be narrowed to a subset of selected final motion features to improve the classification of the user's impairment. Based on the selected motion features, the user's motion features are compared to those same or similar motion features of a healthy population and/or impaired population for ranking the difficulty of individual tasks. The deviation from a centroid of the healthy population can provide a difficulty ranking for each of the tasks tested and measured. A series of tasks may then be recommended to the user based on the difficulty ranking.

Additional information about the disclosed systems, methods, and apparatuses, are provided below.

### Example 1—Methods for Classifying the Severity of Impairment

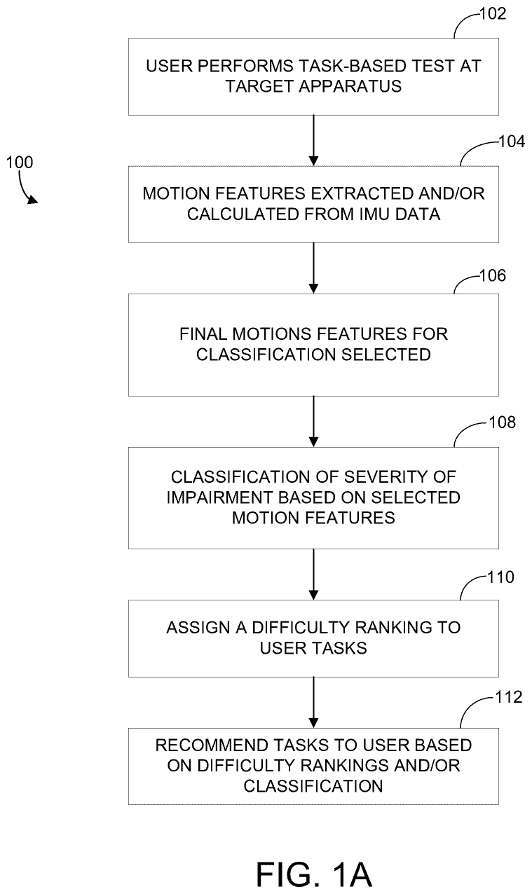

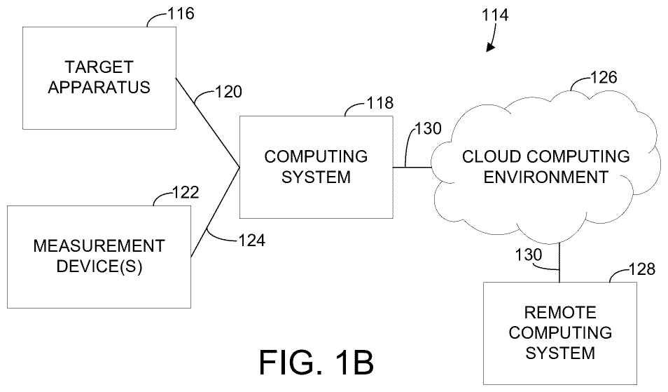

FIG. 1 is a flowchart depicting an example method 100 for classifying the severity of an individual's impairment and providing a series of tasks ranked according to their difficulty. At 102, a user performs a task-based test. The task-based test can include having a user interact with a target apparatus that forms a three-dimensional space that the user moves within and around as they perform one or more tasks included in the task-based test. The tasks can be prescribed in the physical three-dimensional world, meaning the user is expected to interact with the objects and targets of a physical target apparatus, or tasks can be implemented in a virtual or augmented reality environment in which the user interacts with a computer-generated target apparatus displayed through a visual interface. The task-based test can include directing the users to perform a variety of physical tasks associated with the target apparatus while measuring each of the user's movements of interest via one or more inertial measurement units (IMUs). However, other methods of measurement can be utilized to measure user movement. For instance, in addition to or in lieu of the IMUs, alternative wearable devices can be used, including but not limited to near-field sensors, radar-based sensing systems, and/or non-contact motion measurement methods such as marker-based or markerless video capture systems. Each task can emulate common, everyday actions, but may include more complex actions. For example, users may be asked to reach or point to a target, grab and/or lift on object from a target platform, turn a target (e.g., a doorknob), or to perform a variety of other tasks. The target apparatus can have a plurality of targets arranged and coupled to its target structure where each target corresponds with a task of the task-based test. In this way, the target apparatus can provide an adaptable and consistent measurement environment for all users. Additional details regarding target apparatus are provided in further examples below.

At 104, motion features associated with the user's movement and measured during the task-based test are extracted and/or calculated from the data collected via the one or more IMUs and/or other measurement devices. Such data collected can be referred to as user data and can be output to a computing system and/or cloud computing environment described herein for extraction, calculation, and classification of the motion features. Each of the motion features measured during a performed task can be grouped and associated with that particular task. The motion features can be a variety of kinematic and kinetic motion features that are correlated with impairment severity and associated with one or more parts of the user's body (e.g., Example 3). Parts of the body selected for measurement can include, for example, the torso, upper extremity, and/or lower extremity. Selection of the body parts used for measurements can be a determination made by a medical professional, a user, and/or other selection process. One or more motion features may also be measured by one or more optical devices used in conjunction with the IMUs and/or other measurement devices.

The motion features measured can be any number of motions features which are observable and/or measurable via the measurement devices. The motions features can serve as an input used in a machine-learning algorithm or system to classify the severity of the user's impairment and/or to provide recommended tasks. The motion features can also be communicated to a remote computing device monitored by a medical professional, individual, or group, who may also choose to modify the set of motion features measured and/or input for classification. The remote computing device can be a variety of devices, including any handheld, smart device, or processing unit, which includes software a medical professional uses to interact with the patient (e.g., via telemedicine web-based and/or cloud-based application) and to monitor the user's impairment severity and/or progress.

At 106, a final set of motion features can be selected or extracted for use in a machine-learning algorithm or system for classification. The final motion features can be a narrowed subset of the total number of motion features measured. The final motion features can include final motion features for each task the user performs, such that for each task completed by the user during the task-based test, the user's severity of impairment can be classified.

The final motion features used for severity classification for a given task can be predetermined via a feature selection process of the machine-learning algorithm that selects the optimal, or near optimal, motion features for that task's classification. The final motion features can be selected from a set of candidate motion features. The candidate motion features for example, can be features determined or selected based on their potential or usefulness to distinguish users' motion features from corresponding motion features of healthy and impaired populations for one or more given tasks. The candidate features can be selected by a medical professional, an operator, and/or user (e.g., via a local and/or remote computing system). This selection can also be made via an algorithm trained to do as much. The feature selection process can select the final motion features used to classify each task based on modeling of the healthy and impaired classes used to classify the severity of the user's impairment.

At 108, the final motion features from 106 can be used in a machine-learning algorithm or system trained to classify the severity of the user's impairment as one of two or more different classes of severity based on the final motion features. For example, the severity of the user's impairment can be classified or predicted to be within a healthy class or within one of three classes of severity. The three classes of severity can include mild, moderate, and severe, but may be expanded to include one or more discrete classes of severity. Each severity class for classification can be based on motion features from a population representative of that class. The healthy class, for instance, can be based on a collection of motion features from a population of individuals determined to be healthy for the purpose of severity classification. For example, the healthy population can include individuals that have no history of impairment and/or are determined to be healthy by one or more metrics, e.g., the FMUE scoring system. Likewise, the mild, moderate, and severe severity classes can be based on motion features from populations that have been determined and/or known to be mildly, moderately, and severely impaired, respectively. The classification of the severity of impairment can include first classifying the severity of the user's impairment based on each completed task. In this instance, the final severity classification can be based on the average classification across all or a number of the classifications for the completed tasks.

At 110, the difficulty of each task completed by the user can be ranked, or in other words, each of the completed tasks can be assigned a difficulty ranking. Based on the difficulty ranking, one or more of the completed tasks can be recommended to the user at 112 as part of a rehabilitation plan. Each of the completed tasks can be ranked according to its difficulty for the individual user. The difficulty of any given task for an individual user may be measured by how far the user's motions deviate from the motion patterns of the healthy population and/or impaired population. At 112, one or more tasks can also be recommended based on the final severity classification.

The method 100, or any part thereof, may be repeated any number of cycles as part of a broader, ongoing healthcare strategy such that the severity of the user's impairment can be classified, and tasks recommended on a continual basis. The method 100 and the relevant portions thereof, also allow for the motion features, classifications, and/or recommended tasks to be modified by a medical professional as the professional sees appropriate.

FIG. 1B is a schematic diagram depicting a system 114 for classifying the severity of an individual's impairment and recommending user tasks. As illustrated in FIG. 1B, the system comprises a target apparatus 116 that forms a space which a user moves within and around as they perform one or more tasks included in a task-based test. As will be further described herein, the target apparatus 116 (e.g., target apparatus 200) can be a physical target apparatus, displayed within a virtual reality, and/or displayed as an augmented reality enhancement. A computer-generated target apparatus 116 can, for instance, be provided via the computing system 118 at 120 (e.g., computing system 700).

The system 114 also comprises one or more measurement devices 122 (e.g., wearable sensors). The measurement devices 122 are operable to measure the user's movement when the user is performing the task-based test. At 124, the data collected via the measurement devices 122 can be communicated to the computing system 118 and/or a cloud computing environment 126 (e.g., via the network 130). This data can be used in the machine-learning methods described herein for classifying user impairment and recommending tasks based on a difficulty ranking. The computing system 118 can also be used to observe impairment classifications and/or task recommendations. In some embodiments, a medical profession or other operator can alter or modify a classification and recommended tasks via the computing system 118.

In some embodiments, the system 114 can also comprise a cloud computing environment 126 (e.g., cloud computing environment 800). The cloud computing environment 126 can include one or more remote servers located centrally or distributed, and configured to provide cloud-based services to computing systems 118, 128 via a network 130 (e.g., Internet, wireless network, etc.). The cloud computing environment 126 can perform tasks and/or provide services in addition to or in lieu of the computing system 118. For instance, the cloud computing environment 126 can perform computing operations used in impairment classification and/or task recommendation, and can also provide communication between the computing system 118 and one or more remote computing systems 128 (e.g., remote communication between users and medical professionals). In such embodiments, the remote computing system 128 can be used by medical professionals to observe and/or communicate with users over video and/or audio (e.g., during the task-based test), and/or view or modify impairment classifications and user recommended tasks. A medical professional, for instance, can view or modify impairment classifications and recommended tasks via a web-based and/or cloud-based application. Accordingly, the methods and system described herein can be implemented in a clinical setting and/or as a telerehabilitation service to provide support outside of a physical, clinical setting.

### Example 2—Target Apparatus

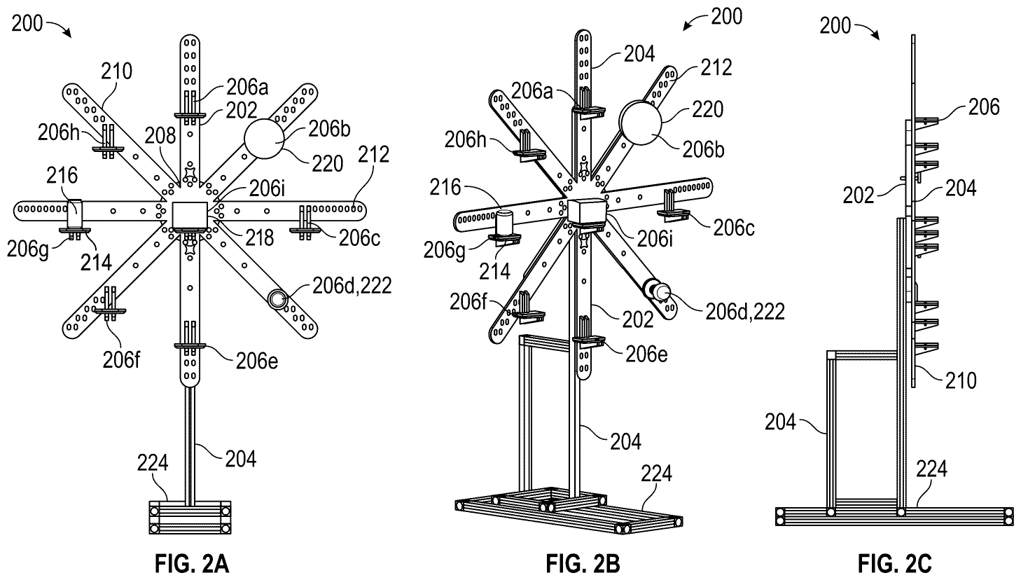

FIGS. 2A-2C depict an exemplary target apparatus. The target apparatus 200 can form a three-dimensional space for users to navigate as they perform a task-based test. In the examples described herein, the user can be positioned facing and at eye level (or near eye level) with the central portion of the target apparatus 200. In this way, the user moves from a central position and is directed to perform a variety of tasks using one or more extremities within the space of the target apparatus 200 during the task-based test. The size of the target apparatus 200 can be scaled for desktop applications and/or for standing applications, and the user can be positioned in any number of ways to interact with the target apparatus.

As shown in the illustrated embodiment, the target apparatus 200 comprises a base 204, a target structure 202 coupled to the base 204, and a plurality of targets 206a-206i coupled to the target structure 202. The target structure 202 can comprise a central portion 208 and a plurality of arms 210 that radially extend from the central portion 208. The arms 210 can also be circumferentially spaced around a circumference or perimeter of the central portion 208. In this configuration, the arms 210 and central portion 208 can be said to form a spoke-like structure. Yet, the target structure 202 can have a variety of different forms and/or configurations so long as the targets 206 can be coupled (and decoupled) and arranged in at least two-dimensional space about the target structure's center (e.g., central portion 208). In some embodiments, the targets 206 can be coupled to the target structure in any arrangement.

Each of the targets 206a-206i of the target apparatus 200 can be associated with a task of a task-based test. The targets 206a-206i, for example, can have one of a variety of configurations such that each target can be configured for a particular task. As shown in the illustrated embodiment of FIGS. 2A-2C, one or more targets 206 (e.g., 206a, 206c, 206e-206i) can be configured with a platform 214 for one or more objects to be placed on. The platforms 214 of targets 206g and 206i, for instance, are sized and shaped to support a container 216 (e.g., soda can) and a box 218, respectively. The container 216 and the box 218 can be associated with any number of grabbing and/or lifting tasks to be performed as part of the task-based test. The container 216, for example, can be associated with a grabbing or lifting task that directs the user to lift the container with one upper extremity, while the box 218 can be associated with a similar grabbing or lifting task that directs the user to use both upper extremities to perform and complete the task.

As shown in FIGS. 2A-2C, one or more of the targets 206 can have a configuration different from that of the platform 214. In particular, target 206b and target 206d, can be configured as a disc or pad 220 and/or a doorknob 222, respectively. The pad 220 for example can act as a reaching or pointing target in which the user is directed to contact one or more points on the surface area of the pad 220. The doorknob 222 by contrast, directs the user to make contact, grab, and turn the doorknob. In some embodiments, two or more of the targets and the tasks thereof, can form a single task of the task-based test. For example, directing the user to touch the pad 220 with one upper extremity while lifting the container 216 with the other.

In some embodiments, one or more of the targets 206 comprise one or more sensors (not shown) that can be used to identify whether a task has been performed, completed and/or to measure the user's performance of the task. In such embodiments, each of the sensors are in communication with a computing system (e.g., computing system 700) such that the computing system can determine whether and when a task has been completed and/or to measure performance of the task. For instance, one or more of the platforms 214 can be configured with one or more sensors that indicates to the computing system and/or a local observer (e.g., via an optical signal) when the user has lifted, grabbed, or shifted, one or more objects placed on the platform 214. Such sensors can include a motion sensor and/or one or more load cells to indicate whether the object is positioned over or on the platform. The objects placed on the platforms 214 (e.g., the container 216 and box 218) can also have one or more sensors, including each of the sensors included in the IMUs. For example, an accelerometer, gyroscope, and magnetometers for measuring the orientation and movement of the object as the user manipulates the object.

The pad 220 can have one or more motion sensors or touch sensors for detecting movement of the user's upper extremity or contact made by the user. Similarly, the doorknob 222 can include a torque and/or rotational sensor/meter to determine whether the user fully completes the task (e.g., turns it one full rotation) and to measure the amount of torque and rotation the user applies to the doorknob 222. Although the targets 206 and one or more sensors are described herein with particularity, the targets 206 and respective sensors can be configured in a variety of manner in accordance with the principles discussed herein to provide a number of alternative targets and associated tasks.

In the illustrated embodiment, the central portion 208 and each of the arms 210 comprise a plurality of apertures 212 configured to receive one or more targets 206. The apertures 212 can be sized and shaped to receive one or more protrusions of the targets 206. In particular, each of the targets 206 can have one or more hooks that extend through a corresponding number of the apertures 212 such that each of the targets 206 are retained by a respective arm 210. In some embodiments, the targets 206 can include one or more pegs, pins, bolts, screws, and/or other protrusions to be received by the apertures 212. In other embodiments, each of the targets 206 be coupled to a respective arm 210 via an adhesive, hook-and-loop portions, a magnetic device, and/or other forms of coupling. This, among other things, allows each of the targets 206 to be positioned along the length of each of the arms 210 such that the targets 206 can be positioned at various distances and radially from the central portion 208 and the user. The positioning of each of the targets, for example, can be positioned to accommodate the handedness of the user, and/or be used to measure particular user movement, such as those tasks where its preferable to have the user fully extend an upper extremity to complete a task.

Each of the targets 206 and the target structure 202 can also include a variety of additional features. For instance, the arms 210 can include one or more optical devices, such as light emitting diodes (LEDs) to direct the user to a particular target, i.e., to a particular task and each successive task. In addition to, or in lieu of the optical device, the arms 210 can include one or more audio devices, such as one or more speakers to direct the user. These “directional” features can, for example, help to direct the user during randomized task-based testing.

Still referring to FIGS. 2A-2C, the distance between the user and target structure 202 can be adjusted via its coupling to the base 204. The base 204 for example is configured to be slidably adjustable relative to the user such that the target structure 202 and targets 206 can be positioned closer to and/or away from the user. In this way, the target structure may be adjusted to vary the difficulty of each task and/or the user's movement in response to the adjustment. The base 204 can comprise a wheel, roller, and/or track system 224 which allows the target structure to move closer to and/or away from the user. In some examples, the target structure 202 is positioned at two or more different distances from the user during the task-based test and can be based one or more anthroprometric measurements of the user. In some examples for instance, the target apparatus can be set at a first distance from the user ranging from 30% to 50% of the user's arm length during testing, i.e., the length from the shoulder to the fingertips. In further examples, the target apparatus can be set at a second distance from the user ranging from 70% to 90% of the user's arm length. Other anthroprometric measurements including the weight, height, or length of the user may be used.

Although the target apparatus 200 is described as a physical, tangible system, it should be understood that the target apparatus can also be displayed to the user via a virtual or augmented environment. For instance, a computer system as described herein can display a computer-generated target apparatus with the same features and functionality as target apparatus 200. The computer-generated target apparatus can, for example, be displayed through a virtual or augmented interactive interface configured to provide perceptual information to the user across one or more sensory modalities, such as visual, haptic, auditory, etc. The user in this case can perform the task-based test and recommended tasks without being in the physical presence of the target apparatus 200. Additionally or alternatively, computer-generated target features can be used in conjunction with one or more tangible features of the target apparatus 200. By way of example, one or more targets (e.g., targets 206a-206i) can be computer generated and additive to the physical target structure 202 (e.g., overlaying the physical target structure 202) through an augmented reality interface.

The computing system in instances where a virtual and/or augmented reality is utilized can include any one or combination of electronic devices configured to display and/or make interactive the virtual and/or augmented environment, including a headset, mobile device, personal computer, gaming console, controller, sensor, specially manufactured product, etc.

### Example 3—Inertial Measurement Units (IMUs) and Motion Features

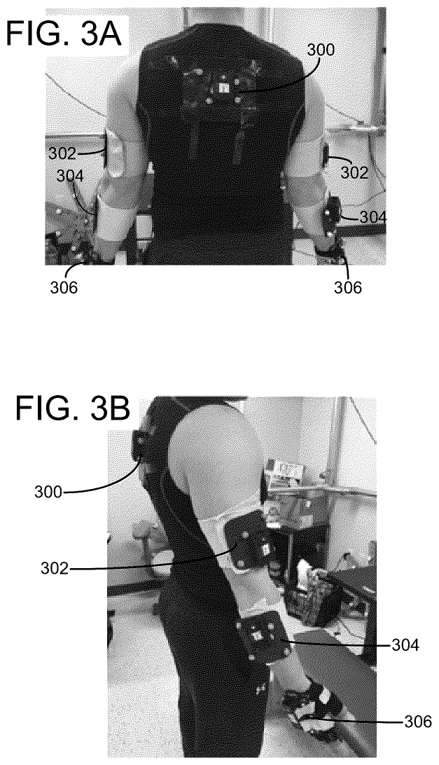

Referring to FIGS. 3A-3B, classifying the severity of user impairment can be based on real-time and/or stored data output by one or more inertial measurement units (IMUs) 300-306 to a computing system (described below). A number of kinematic and kinetic motion features corresponding to the user's movement and measured during the task-based test via the one or more IMUs can be extracted and/or calculated from the data collected. The motion features can, for example, be extracted and/or calculated by a computing system and used as inputs for severity classification. In some examples, the time it takes a user to complete a task can be associated with the motion features for that task. The time for task completion can be calculated via the IMUs and/or computing system.

The IMUs 300-306 can be wearable IMUs or otherwise attachable to the user to collect the user data associated with the motion features. As shown in FIGS. 3A-3B, for example, IMUs 300-306 can be located at the torso (e.g., chest), upper arm, forearm, and hand, respectively, to capture the dynamic motions of the user (e.g., of the shoulder, elbow, and wrist joints) as the user engages with a target apparatus (e.g., target apparatus 200). In some examples, the IMUs 300-306 can be positioned and secured to the user at one or more of the upper extremities, lower extremities, and/or torso. In some examples, the IMUs can be incorporated into a unitary garment, such as a shirt, pants, and/or other garment that comprises a network of individual and/or interconnected IMUs.

The IMUs can measure in three orthogonal directions, acceleration, angular velocity, and magnetic field, of the body parts to which the IMUs are attached using a combination of accelerometers, gyroscopes, and magnetometers. The data collected from the IMUs can be used to determine the relative position, orientation, and velocity of a rigid body part (e.g., an upper arm segment) in a fixed reference frame without requiring physical attachment to a ground surface. This, among other things, makes IMUs particularly well-suited for wearable sensor applications, such as providing feedback for rehabilitation, because the patient can wear a garment in which the sensors are embedded. In this way, the IMUs, can be a portable means to collect user data associated with the motion features. As such, in the examples described herein, the IMUs are capable and configured to collect sufficient data for the classification and task difficulty ranking without optical equipment for measurement. In some examples, in addition to or in lieu of the IMUs, alternative wearable devices can be used for data collection, including but not limited to near-field sensors, radar-based sensing systems, and/or non-contact motion measurement methods such as marker-based or markerless video capture systems.

Nevertheless, in some examples, one or more optical tracking systems can be used in conjunction with one or more IMUs to collect the user data during the task-based test. Such optical tracking systems may be passive (e.g., marker based) and/or active systems (e.g., optical devices emitting light a predetermined frequencies). In such examples, the optical tracking system can enhance or simplify data collection, by offsetting the number IMUs used, and/or can be used to compensate fora misalignment and/or other mispositioning of one or more IMUs secured to the user. In some examples, the optical devices can be a device such as a handheld, smart device, gaming console, and/or other processors with sufficient optical capabilities.

As mentioned above, the user data collected via the IMUs can be used to determine a variety of kinematic and kinetic motion features that are correlated with the severity of impairment. Such motion features can be extracted and/or calculated by a computing system, or operable IMUs. Dynamic joint kinematic motion features, for instance, can be measured at precise or near precise quantities using the IMUs, including movement time, velocity, strategy, smoothness, and inter-joint coordination which all can be used to identify differences between the movement of a healthy population and impaired populations, including the movement of the user.

Kinetic motion features by contrast can give unique outputs (i.e., torques) for sporadic behavior that have a high correlation with impairment severity. For instance, damage to the central nervous system caused by a stroke can be observed in gravity, interaction, focal point, muscle, and/or net torques. As an example, stroke patients are highly dependent on forces from neighboring limbs to complete a task, whereas a healthier individual's net torque demonstrate a higher dependency on muscle and gravity torque from a single joint to generate movement. This distinction can be observed, for example, during a tasked-based test when a user is asked to reach and grab a container or box from one or more of the platforms of the target apparatus. Kinematic and kinetic motions can also be used to observe dynamic compensatory motor strategies of user movement between starting and ending positions during the task-based test. These compensatory motor strategies often go undetected using typical examining systems and methods.

Examples of motion features that can be used for severity classification can, for instance, include body displacement (e.g., chest displacement), variance of joint angle, zero crossing of a joint angle, a max amplitude of a joint angle velocity, frequency of the max amplitude of joint velocity, the Spearman Correlation between flexion angles, mean absolute phase difference of proximal and distal joints, joint peak velocity time, and/or root mean square of joint acceleration. Torso and shoulder angles (e.g., torso bending, shoulder flexion, shoulder abduction, shoulder rotation, torso flexion, elbow flexion, forearm pronation and wrist angles (wrist flexion and wrist deviation) can also be included. Though a number of motion features are listed, the listed features are not intended to be exhaustive, but representative and may include one or more additional or alternative motion features.

### Example 4—Machine Learning and Training for Classification

A standard for performance-based measure of stroke-specific impairment is the FMUE scoring system. In the FMUE scoring system, each task is given a score of 0 for noncompletion of the task, 1 for partial completion, and 2 for full completion (e.g., a total FMUE score of 66 generally).

Once the motion features have been extracted, calculated, and/or received from the IMUs, the motion features can be used in a machine-learning process to provide a classification that relates the motion features to the FMUE score. The methods of the present disclosure can map the kinematic and kinetic motion features of various populations (e.g., healthy and impaired) to the FMUE score to train a machine-learning algorithm or system (e.g., artificial intelligence) to create an automated system that can classify the severity of an individual user's impairment based on the user's motion features. For instance, a full scale of the FMUE score (0-66) can be mapped to the kinematic and kinetic motion features, and machine-learning based classification can be made at increasing levels of resolution similar that of the FMUE scale and, in some instances, with greater accuracy than current clinical methods. Although the FMUE scoring system is used herein as a specific example, any other evaluative method and/or scale can be used in accordance with the techniques and principles described herein. Such evaluative methods may be used for a number of different applications and impairments, such as those resulting in orthopedic and/or non-neurological impairment.

Machine-learning algorithms or systems as described herein may be any machine-learning algorithm that can be trained to provide improved results or results targeted to the classification of the severity of impairment. Types of machine-learning can include supervised learning, unsupervised learning, neural networks, classification, regression, clustering, dimensionality reduction, reinforcement learning, and Bayesian networks.

Training data, as described herein, refers to the input data used to train a machine-learning algorithm so that the machine-learning algorithm can be used to analyze “unknown” data, such as the user data associated with one or more motion features measured during a task-based test. Testing data can also be part of the training data set. The testing data can represent a desired or expected classification which may be compared with the output from the algorithm when the training data inputs are used, and the algorithm may be updated based on the difference between the expected and actual outputs. Generally, each processing of a set of training data through the machine-learning algorithm is known as an iteration, episode, or a cycle. Training data can, for example, include the motion features from a population or class of individuals. The motion features from a population or class can, for example, be grouped and associated with respective tasks of a task-based test in which those motion features of the population or class of individuals were measured. In this way, a classification of a user's impairment can be made for each task within the task-based test performed. A population or class, in this case, can include healthy, mild, and moderately or severely impaired individuals.

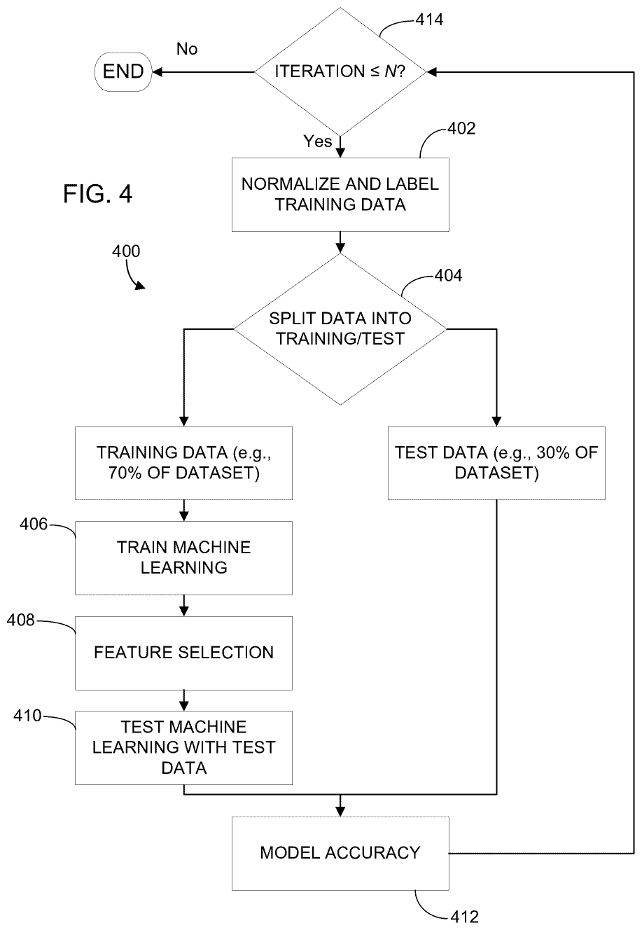

FIG. 4 is a flowchart depicting an example method 400 for training or developing a machine learning algorithm or system that is operable to classify the severity of an individual user's impairment based on the user's motion features. At 402, normalized techniques can be applied to training datasets. Such techniques can include, but are not limited to, min-max, mean, and/or z-score normalization.

Also, at 402, reference FMUE scores can be used to label the normalized data for training and testing the machine-learning algorithm. For example, reference FMUE scores used in labeling the normalized data can be provided or based on clinicians' independent monitoring of those populations or classes whose motions features are used within the training and testing data (e.g., healthy and impaired populations). In some examples, other labels can be utilized.

The machine-learning algorithm can be one of a variety of algorithms used for classification, for instance, a perceptron, Bayesian, logistic regression, K-nearest neighbor, neural network, deep learning, and/or support vector machine algorithm. By way of example, a support vector machine (SVM) algorithm can create an optimal hyperplane and optimal boundaries (or support vectors) between the motion features of different severity classes using the labeled datasets which results in the maximizing classifier margin and classification score. One or more kernel methods may also be used to linearly separate data that is nonlinear, such as by mapping the nonlinear separable data into a higher dimensional space. A number of kernel methods can include a linear, polynomial, Sigmoid, radial basis function kernel, Fisher, string, neural network Gaussian process, and/or a combination thereof.

As shown at 404, the training data set can be split into training data (e.g., 70%) and testing data (e.g., 30%). At 406, the SVM can be used to model three classes from the training data: healthy (e.g., a FMUE score of 66), mild (e.g., 66>FMUE score≥47), and moderate or severe (e.g., FMUE score of <47). For instance, multi-class classification techniques can be used to categorize the training data into the multiple class labels, such as via One-vs-Rest and One-vs-One classification. As such, the data can be split into multiple binary classification datasets and each combination of classes can be classified. For instance, the three classes can be split into binary combinations including healthy vs. mild, healthy vs. moderate or severe, and mild vs. moderate or severe. As such, the machine-learning algorithm can be configured to output a machine learning model comprising the model data and provide a classification prediction for a given user. In some embodiments, sampling techniques can be used to reduce potential disparities between class sizes and to improve classification accuracy.

As shown at 408, a feature selection process of the method 400 can be used to find an optimal subset of features for the classification model, i.e., to find a final subset of motion features for each task that can be used to accurately classify the severity of users' impairment for each of those tasks. The feature selection process can be an iterative process to search for a set of features that maximize the accuracy of the prediction process for a given task.

The feature selection process can, for example, begin by determining a set of candidate features for each or a given number of tasks of the task-based test. The candidate features can be a subset of the total features that are measurable via the IMUs. The candidate features can include any number of motion features that are chosen based on their potential to be used in the classification process of severity of impairment, such as those motion features that can distinguish a healthy population from users with varying degrees of impairment for a given task (e.g., see list of features in Example 3). Such distinguishing candidate features, for example, can be determined by a comparison of kinematic and/or kinetic profiles of members of the different impairment severity classes. Selection of the candidate features can be determined based on a variety of criteria, including particular metrics and/or the comprehensive nature of the motion features. For instance, the candidate features can include those motion features that include each of a number of specified joint angles, the captured range of motion of a number of specified joints, and/or the captured time-based and/or range-based relationship between joint motions. The creation of the candidate features can be determined by a medical professional, operator, and/or algorithm based on one or more criteria.

The predictability of severity of impairment using the candidate features can be tested via various feature selection methods, such as wrapper, filter, and/or embedded methods, which can narrow the candidate features to a final subset of features. For example, a Sequential Forward Floating Selection (SFFS) method can be used for feature selection to select from the candidate features the final subset of features used for classification. In other words, a list of the motion features the machine-learning algorithm will look for in a user's motion features to classify the severity of impairment for a given task.

The SFFS for instance, begins with the creation of an empty set of features from the candidate features and adds one candidate feature at a time until a predetermined number (e.g., determined by an operator or algorithm) of selected features are obtained that optimizes the selection model accuracy. The SFFS, includes an exclusion step that discards a candidate feature only if removal of that candidate feature increases the selection model performance. This process is repeated until a predetermined number of selected features, the final subset of features, are found for each task. The predetermined number of selected features can be any number of features determined by a medical professional, an operator, and/or algorithm for the purpose of classification.

At 410 and 412, the test data (e.g., the 30%), can be used to test the performance and accuracy of the classification model using the final subset of features determined from the selection process. This process can be an iterative process to test the performance and accuracy of classification of severity for each task of the task-based test. The performance and accuracy can be determined by a variety of performance metrics. By way of example, an F1 score calculated from both the precision (consistency of the prediction) and recall (reliability) of the classification model can be a measure of the model accuracy. The F1 score can be a measure by which to determine whether the final subset of features from the feature selection process in the classification model results in the classification model meeting an accuracy threshold. If the resulting F1 score threshold (e.g., F1 score 0.90) is not met, the least valuable features of the final subset of features are discarded, and new or additional candidate features can be considered by the feature selection process. The new or additional features can be determined by an operator and/or algorithm. Yet, if the resulting F1 score threshold is met, the selected features can be selected as the final subset of features for input in the classification model by which to classify a user's motion features.

The overall performance and accuracy of the classification model can be determined by averaging the F1 scores through each iteration N. This, among other things, can determine which kernel methods provide the highest classification accuracy, for example, when an SVM algorithm is used. An overall F1 score of 80%, 85%, 90%, 95%, or higher may be used as a threshold that indicates the accuracy of the classification model has met the level of accuracy wanted for the severity of impairment classification.

The training process can, for example, be executed for any N number of iterations. The number of iterations N can be determined via an operator, medical professional, and/or algorithm. At 414, the machine-learning algorithm determines whether the current iteration is less than or equal to N. If the current iteration is greater than N, the training method ends. Alternatively or additionally, the training can be prescribed to end when a certain threshold of performance or when a certain performance measure, such as a certain F1 score, reaches a diminishing improvement below a given threshold.

### Example 5—Classification

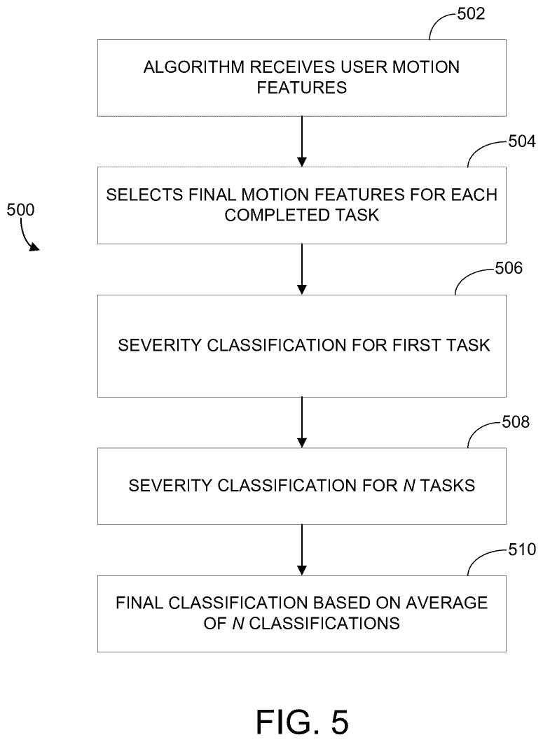

Once the machine-learning is trained and meets an accuracy threshold, the motion features measured during the user's task-based test can be used by the machine-learning algorithm to classify the impairment severity of the user. FIG. 5 is flowchart depicting an example method 500 for classifying the severity of an individual's impairment using machine-learning algorithm tools. At 502, the machine-learning algorithm receives the motion features associated with the user's movement measured and collected during the task-based test (e.g., via the IMUs 300-306 and/or computing system 700). From these user motion features, at 504, the machine-learning algorithm selects those motions features determined to be the optimal (or near optimal) motion features for classification for each of the user's completed tasks. For example, for a given task, the machine-learning algorithm selects the user's motion features corresponding to the final subset of features that were determined to be optimal for severity classification for that particular task during the feature selection process (e.g., of method 400).

At 506, to classify the severity of the user's impairment, the selected features from the user's motion features for a first given task are evaluated against the motion features associated with the same task (or similar task) from the three classes of severity, i.e., for healthy, mild, and moderate/severe populations. Based on the evaluation, a prediction or classification of the severity of impairment is made for the first task being evaluated. As such, at 508, the process of classification can be completed for each or N number of the tasks completed by the user during the task-based test. The final classification of the severity of impairment in this case, or classification output of the machine-learning algorithm, can be the average or weighted average classification across the total number of classifications for the task-based test at 510. In some examples, the final classification is the average classification across only a specified number of classifications and/or classifications for only particular tasks (e.g., determined by medical professional, operator, and/or algorithm).

In some examples, a voting ensemble machine learning model (e.g., a hard voting and/or soft voting) can be used to sum the predictions made by the multi-classification model. For example, for each given task, the class (e.g., healthy, mild, or moderate/severe) with the highest sum or “vote” (or predictions) establishes the final severity classification for that task. This, among other things, allows the multi-classification model to achieve better performance by classifying the severity of the user's impairment using a combination of modeling techniques (e.g., kernals). Although a voting ensemble is mentioned, a variety of voting models can be used, including stacking, boosting, and/or bagging.

In some examples, the classification of the severity of the user's impairment is communicated via the computing system to a remote computing device for a medical professional and/or other individual to monitor and/or modify the final severity classification or classification for any number of tasks. Any modified classifications can be transmitted or otherwise communicated to the user or medical professional. Modifications to the final classification or classification of tasks can also be communicated to the machine-learning algorithm such that the modified classifications can be used in assigning a difficulty ranking to tasks and/or otherwise recommending tasks to the user.

### Example 6—Difficulty Ranking

After the severity of the user's impairment has been classified, a hierarchy of tasks may be recommended to the user as part of a personalized rehabilitation plan in an effort to help the user regain mobility. To regain mobility, the rehabilitative goal is to have the user regain motion patterns similar or the same as those of the healthy population. Similar to those method described above, the methods described herein can be implemented using machine-learning tools such that output of machine learning can provide the difficulty ranking and recommendations to the user and/or a medical professional.

Each of the tasks completed by the user can be assigned a difficulty ranking according to that task's difficulty to the user. Based on the difficulty rankings, one or more of the completed tasks can be recommended to the user as part of a rehabilitation plan. The difficulty of any given task for a user can be measured by how far the user's motion features deviate from the motion features of the healthy population for the same task (or similar task). A numerical value can be assigned to the deviation and correspond to the task's difficulty ranking. For instance, the numerical value can correspond to a value within a scale of numerical values used to classify a task's difficulty, e.g., the FMUE scale. Alternatively or additionally, the difficulty can be determined by the Euclidean distance of a user's task classification in the machine-learning feature space to that of healthy subjects.

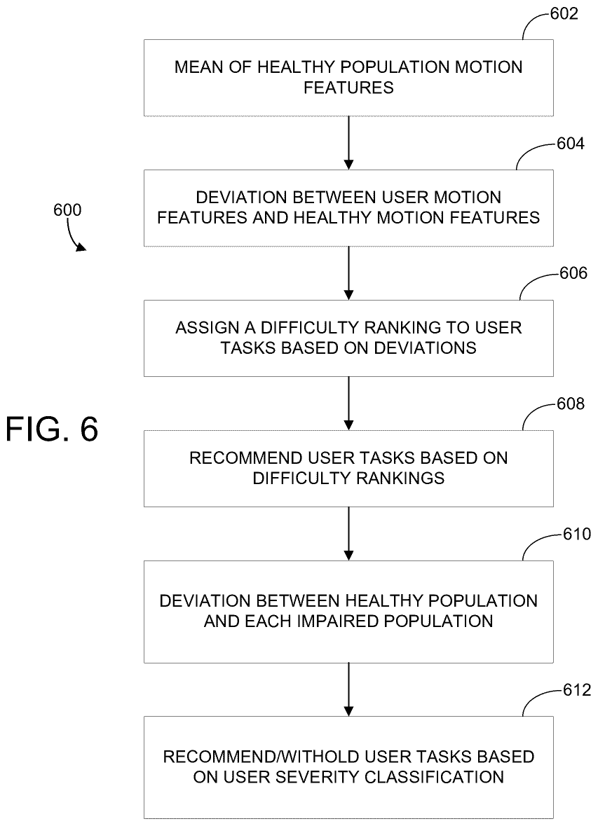

FIG. 6 is a flowchart that depicts an example method 600 for assigning one or more tasks a difficulty ranking and recommending a series of tasks based on those difficulty rankings. The feature space for ranking the difficulty of any individual task can be defined by the final subset of features used to classify the severity of impairment for that given task (e.g., from the feature selection process of method 400 and selected final features in method 500). However, other motions features may define the feature space.

At 602 and 604, the motions features that correspond to the final subset of features from the healthy population for a given task can be used to calculate a Euclidean distance or deviation between the healthy population and the user's motion features. For instance, the centroid or mean of the healthy population's motion features for a given task can be used to measure the Euclidean distance between the healthy population and the user's motion features for the same task. The deviation between the healthy population and the user's movements can be used to assign a difficulty ranking to the given task for the user at 606. The deviation for instance, can be assigned a numerical value that corresponds with a difficulty ranking, such as by ranking the tasks on a scale with the relatively greatest deviation as the most difficult, and ranking those tasks with the relatively least deviation as the easiest for the user to complete. Tasks the user is unable to complete, and thereby the data may not be assessable because of incomplete motion data, can be assigned an arbitrarily high Euclidean distance, resulting in the uncompleted tasks being ranked with the relatively most difficult tasks. This difficulty ranking can be used to determine which tasks to recommend to the user 608. As one example, each or a number of the tasks determined to have been completed by a user with only a relatively minor deviation from healthy performance (similar to being scored as partially or fully completed by the user on the FMUE scale (e.g., FMUE score of 1-2, respectively) can be recommended to the user as part of a rehabilitation plan. In this case, those tasks the user failed to complete (similar to a FMUE score of 0) or completed with relatively high difficulty (e.g., relatively high Euclidean distance) would not be recommended, but may be recommended later as part of rehabilitation strategy or as the user gains healthier mobility.

At 610, a Euclidean distance or deviation between the healthy population and each of the impaired populations (e.g., mild and moderate/severe) can establish a general difficulty ranking or baseline for a number of tasks, which can be used to modify the tasks recommended to the user at 608. As one example, the deviation between the mildly impaired population and the healthy population may indicate that overall, those within the mildly impaired population are generally unable to complete a number of specified tasks. Accordingly, at 612, those specified tasks can be withheld from being recommended to those users whose severity was classified as mild, whether or not the user attempted those tasks during the task-based test. Likewise, a deviation between the mildly impaired and healthy populations may indicate that overall, those within the mildly impaired population are able to complete, e.g., with relatively minor difficulty, other certain tasks. At 612, these certain tasks can be recommended to those users classified as mildly impaired, with or without attempting those tasks during the tasked-based test.

Accordingly, a user may receive recommended tasks as part of the rehabilitation plan based on their severity classification, completed tasks (or noncompleted tasks), their individual quantification of task difficulty (i.e., difficulty ranking), or any combination of these.

The recommended tasks can be part of a rehabilitation plan that is monitored by a medical professional. As such, the medical professional can modify the rehabilitation plan via the computing system or a remote device in communication with the computing system (e.g., via network, cloud computing environment, etc.). For instance, via a medical professional's input, one or more of the tasks recommended to the user can be modified, such as by removing tasks, adding tasks, and/or otherwise modifying the performance of the tasks (e.g., changing the degree of difficulty of a task). The modified recommended tasks can then be transmitted or otherwise communicated to the user as part of a modified rehabilitation plan. The method 600 can also be an iterative one, such that new or alterative tasks can be recommended as the user's mobility improves and/or declines. Such recommendations can be made by a medical professional's input based on feedback from the algorithms presented herein, or the recommendations can be automatically generated by the algorithms as user's performance changes, for instance, as the user's performance of tasks gets relatively closer to healthy performance (e.g., by a predetermined threshold).

### Example 7—Example Computing Systems

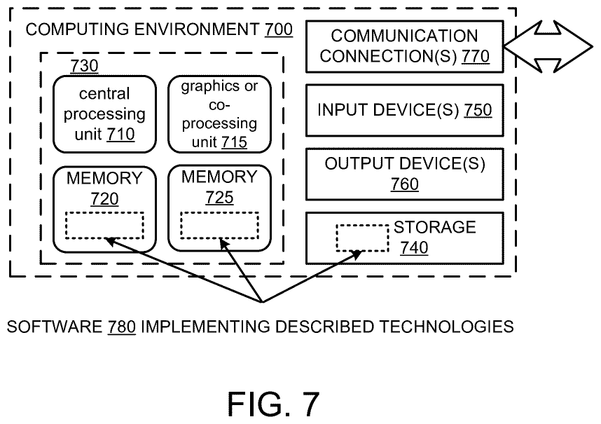

FIG. 7 depicts a generalized example of a suitable computing system 700 in which the described innovations can be implemented. The computing system 700 is not intended to suggest any limitation as to scope of use or functionality, as the innovations may be implemented in diverse general-purpose or special-purpose computing systems.

With references to FIG. 7, the computing system 700 includes one or more processing units 710, 715 and memory 720, 725. In FIG. 7, this configuration 730 is included within the dashed line. The processing units 710, 715 execute computer-readable instructions, such as for implementing the processes of FIGS. 1 and 4-6. A processing unit can be a general-purpose central processing unit (CPU), processor in an application-specific integrated circuit (ASIC) or any other type of processor. In a multi-processing system, multiple processing units execute computer executable instructions to increase processing power. For example, FIG. 7, shows a central processing unit 710 as well as a graphics processing unit or co-processing unit 715. The tangible memory 720, 725 may be volatile memory (e.g., registers, cache, RAM), non-volatile memory (e.g., ROM, EEPROM, flash memory, solid state drives, etc.), or combination of the two, accessible by the processing unit(s). The memory 720, 725 stores software 780 implementing one or more innovations described herein, in the form of computer-executable instructions suitable for execution by the processing unit(s).

A computing system can have additional features. For example, the computing system 700 includes storage 740, one or more input devices 750, one or more output devices 760, and one or more communication connections 770. An interconnection mechanism (not shown) such as a bus, controller, or network interconnects the components of the computing system 700. Typically, operating system software (not shown) provides an operating environment for other software executing in the computing system 700, and coordinates activities of the components of the computing system 700.

The tangible storage 740 may be removable or non-removable, and includes magnetic disks, magnetic tapes or cassettes, solid state drives, CD-ROMs, DVDs, or any other medium which can be used to store information in a non-transitory way and which can be accessed within the computing system 700. The storage 740 stores instructions for the software 780 implementing one or more innovations described herein.

The input device(s) 750 may be a touch input device such as a keyboard, mouse, pen, or trackball, a voice input device, a scanning device, or another device that provides input to the computing system 700. For video encoding, the input device(s) 750 may be a camera, video card, TV tuner card, or similar device that accepts video input in analog or digital form, or a CD-ROM or CD-RW that read video samples into the computing system 700. To measure user movement, the input device(s) 750 can also include one or more inertial measurement units (IMUs), near-field sensors, radar-based sensing systems, and/or non-contact motion measurement systems such as marker-based and/or markerless video capture systems. Moreover, the input devices(s) 750 can include one or more sensors or other devices, such as those included with the targets described herein (e.g., targets 206a-206i). The sensors can include any one or combination of optical sensors, motion sensors, touch sensors, torque sensors, rotational sensors, load cells, accelerometers, gyroscopes, and/or magnetometers.

The output device(s) 760 may be a display, printer, speaker, CD-writer, or another device that provides output from the computing system 700. For instance, one or more displays, speakers, controllers, and other devices can be configured to output computer-generated perceptual information, such as virtual and/or augmented environments. Such devices can also act as input devices. The output device(s) 760 can also include a transducer, or like device, configured to output data/information/energy to direct one or more optical devices, such as light emitting diodes (LEDs) on a physical target apparatus to light up and indicate one or more tasks to be performed.

The communication connection(s) 770 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, RF, or other carrier.

The innovations can be described in the general context of computer-executable instructions, such as those included in program modules, being executed in a computing system on a target real or virtual processor. Generally, program modules include routines, programs, libraries, objects, classes, components, data structures, etc. that perform particular tasks or implement particular abstract data types. The functionality of the program modules may be combined or split between program modules as desired in various embodiments. Computer-executable instructions for program modules may be executed within a local or distributed computing system. Moreover, the disclosed technology can be implemented through a variety of computer system configurations, including personal computers, handheld devices, tablets, smart phones, headsets, multiprocessor systems, microprocessor systems, microprocessor-based or programmable consumer electronics, network PCs, minicomputers, mainframe computers, and the like.

### Example 8—Cloud Computing Environment

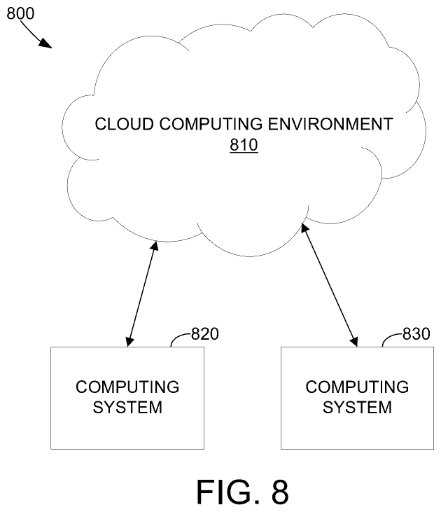

FIG. 8 depicts an example cloud computing environment 800 in which the described technologies can be implemented. The cloud computing environment 800 comprises cloud computing services 810. The cloud computing services 810 can comprise various types of cloud computing resources, such as one or more remote computer servers, data storage repositories, telecommunication resources, networking resources, etc. The cloud computing services 810 can be centrally located (e.g., provided by a data center of a business, organization, or institution) or distributed (e.g., provided by various computing resources located at different locations, such as different data centers and/or located in different cities or countries).

The cloud computing services 810 are utilized by various types of computing systems (e.g., client computing devices), such as computing systems 820 and 830 (e.g., computing systems 118 and 128 described herein). For instance, the computing systems (e.g., 820 and 830) can be computers (e.g., desktop or laptop computers), mobile devices (e.g., tablet computers or smart phones), gaming consoles, or other types of computing devices. For instance, the computing systems (e.g., 820 and 830) can utilize the cloud computing services 810 to perform computing operations (e.g., data processing, data storage, and the like) such as the machine-learning methods for impairment classification and tasks recommendations described herein.

In view of the many possible embodiments to which the principles of the disclosed technology may be applied, it should be recognized that the illustrated embodiments are only preferred examples and should not be taken as limiting the scope of the disclosure. We therefore claim all that comes within the scope and spirit of the amended claims and their equivalents.

1. A system for classifying a severity of a user's impairment, the system comprising:
a target apparatus comprising a base, a target structure coupled to the base, and a plurality of targets coupled to the target structure, wherein each of the targets are associated with one or more user tasks;
a plurality of inertial measurement units configured to collect data associated with a user's movement as the user performs one or more of the user tasks; and
a processor including computer-readable instructions, wherein by executing the instructions the processor is configured to classify a severity of the user's impairment based on the data collected from the inertial measurement units.

2. The system of claim 1, wherein by executing the instructions the processor is configured to assign a difficulty ranking to one or more user tasks performed by the user based on the data collected from the inertial measurement units, and wherein by executing the instructions the processor is configured to recommend one or more user tasks based on the difficulty rankings assigned.

3. The system of claim 2, wherein by executing the instructions the processor is configured to transmit the recommended tasks to a remote device, the remote device being configured to modify one or more of the recommend tasks based on an input from an operator of the remote device to form one or more modified recommended tasks and communicate the modified recommended tasks to the processor.

4. The system of claim 1, wherein by executing the instructions the processor is configured to transmit the classification of the user's impairment to a remote device, the remote device being configured to modify the classification based on an input from an operator of the remote device to form a modified classification and communicate the modified classification to the processor.

5. (canceled)

6. The system of claim 1, wherein by executing the instructions, the processor is configured to classify the severity of the user's impairment for each task the user performs at the target apparatus and to recommend one or more user tasks based on the classification of severity.

7. (canceled)

8. The system of claim 1, wherein the targets are arranged in a radial configuration.

9. The system of claim 1, wherein one or more of the targets comprises an accelerometer, a gyroscope, a magnetometer, or a combination thereof.

10. The system of claim 1, wherein one or more targets comprise a sensor configured to detect whether the user task associated with the target has been performed.

11. The system of claim 1, wherein the base is slidably adjustable relative to the user such that the base is configured to move toward and away from the user.

12. The system of claim 1, wherein the target structure comprises one or more optical devices, audio devices, or a combination thereof to direct the user to perform a user task of the one or more user tasks.

13. The system of claim 1, further comprising one or more optical tracking systems to collect data associated with user's movement, and wherein by executing the instructions the processor is configured to classify the severity of the user's impairment based on the data collected from the inertial measurement units and the optical tracking systems.

14-15. (canceled)

16. The system of claim 1, wherein one or more of the targets comprise a platform having one or more sensors configured to detect whether an object positioned on the platform has been moved.

17. A method comprising:
classifying a severity of a person's impairment based on one or more motion features associated with the person's performance of one or more tasks at a target apparatus, wherein the motion features are determined from data collected from one or more inertial measurement units.

18. (canceled)

19. The method of claim 17, wherein classifying the severity of the person's impairment comprises classifying the severity of the person's impairment for two or more tasks the person performs at the target apparatus, and wherein the method further comprises determining a final classification of the severity as one or both of an average and a weighted average of the classifications for the two or more tasks.

20. The method of claim 17, further comprising assigning a difficulty ranking to one or more tasks performed by the person at the target apparatus based on the motion features.

21. The method of claim 20, wherein assigning the difficulty ranking to the one or more tasks comprises determining a deviation between the motion features of the person and one or more motion features of a healthy population for the respective task.

22. The method of claim 20, further comprising recommending one or more tasks to the person based on the difficulty ranking.

23. The method of claim 17, wherein the target apparatus comprises a target structure and a plurality of targets coupled to the target structure, each target being associated with one or more of the tasks.

24. The method of claim 17, wherein the method uses one or more machine-learning methods selected from the group consisting of a perceptron, Bayesian, logistic regression, K-nearest neighbor, neural network, deep learning, and a support vector machine algorithm.

25. (canceled)

26. A target apparatus used in assessing physical impairment of a user, the target apparatus comprising:
a track system;
a target structure coupled to the track system and comprising a central portion and a plurality of outwardly extending arms circumferentially spaced along a circumference of the central portion; and
a plurality of targets coupled to the central portion and arms of the target structure, each target being associated with a physical task and configured to couple and decouple to the target structure such that each target can be positioned at various lengths relative to the central portion;
wherein the track system is configured to slidably adjust such that the target structure can be adjusted toward and away from the user.
